SEQUENCE LISTING (28 sequences)

SEQ ID NO: 1 - protein, 28 aa, synthetic construct
KPRRPYTDNYTRLRKQMAVKKYLNSILN

SEQ ID NO: 2 - protein, 6 aa, synthetic construct
KPRRPY

SEQ ID NO: 3 - protein, 28 aa, synthetic construct
KPRRPYADNYTRLRKQMAVKKYLNSILN

SEQ ID NO: 4 - protein, 28 aa, synthetic construct
KPRRPYTVNYTRLRKQMAVKKYLNSILN

SEQ ID NO: 5 - protein, 28 aa, synthetic construct
KPRRPYTDNCTRLRKQMAVKKYLNSILN

SEQ ID NO: 6 - protein, 28 aa, synthetic construct
KPRRPYTDNYTSLRKQMAVKKYLNSILN

SEQ ID NO: 7 - protein, 28 aa, synthetic construct
KPRRPYTDNYTRLRKQIAVKKYLNSILN

SEQ ID NO: 8 - protein, 28 aa, synthetic construct
KPRRPYTDNYTRLRKQMAVNKYLNSILN

SEQ ID NO: 9 - protein, 28 aa, synthetic construct
KPRRPYTDNYTRLRKQMAVKKYMNSILN

SEQ ID NO: 10 - protein, 28 aa, synthetic construct
KPRRPYTDNYTRLRKQMAVKKYLNLILN

SEQ ID NO: 11 - protein, 28 aa, synthetic construct
KPRRPYXXNXTXLRKQXAVXKYXNXILN

SEQ ID NO: 12 - protein, 28 aa, synthetic construct
KPRRPYTSDYTRLRKQMAVKKYLNLILN

SEQ ID NO: 13 - protein, 5 aa, synthetic construct
SYAMS

SEQ ID NO: 14 - protein, 17 aa, synthetic construct
TISSAGGYIFYSDSVQG

SEQ ID NO: 15 - protein, 14 aa, synthetic construct
QGFGNYGDYYAMDY

SEQ ID NO: 16 - protein, 17 aa, synthetic construct
KSSQSLLNSRTRKNQLA

SEQ ID NO: 17 - protein, 7 aa, synthetic construct
WASTRQS

SEQ ID NO: 18 - protein, 8 aa, synthetic construct
QQSYNLLT

SEQ ID NO: 19 - protein, 123 aa, synthetic construct
EVKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSS

SEQ ID NO: 20 - protein, 112 aa, synthetic construct
IELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEIKR

SEQ ID NO: 21 - protein, 5 aa, synthetic construct
SYWMN

SEQ ID NO: 22 - protein, 17 aa, synthetic construct
QIYPGDGDTNYNGKFKG

SEQ ID NO: 23 - protein, 13 aa, synthetic construct
KTISSVVDFYFDY

SEQ ID NO: 24 - protein, 11 aa, synthetic construct
KASQNVGTNVA

SEQ ID NO: 25 - protein, 7 aa, synthetic construct
SATYRNS

SEQ ID NO: 26 - protein, 9 aa, synthetic construct
QQYNRYPYT

SEQ ID NO: 27 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 28 - protein, 108 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

(19) United States
(12) Patent Application Publication
(10) Pub. No.: US 2025/0144138 A1
(43) Pub. Date: May 8, 2025

(54) Chimeric Antigen Receptor Therapies and Vasoactive Intestinal Peptide Receptor Antagonists

(71) Applicant: Emory University, Atlanta, GA (US)

(72) Inventors: Sarwish Rafiq, Dunwoody, GA (US); Sruthi Ravindranathan, Tucker, GA (US); Edmund K. Waller, Atlanta, GA (US); Heather Kelsey Lin, Decatur, GA (US)

(21) Appl. No.: 18/715,543

(22) PCT Filed: Dec. 2, 2022
(86) PCT No.: PCT/US2022/080785

§ 371 Date: May 31, 2024

Related U.S. Application Data
(60) Provisional application No. 63/264,823, filed on Dec. 2, 2021.

(51) Int. Cl. A61K 35/17; A61K 40/11; A61K 40/31; A61K 40/42; A61P 35/00; C07K 14/725; C07K 16/30; C12N 5/0783

(52) U.S. Cl. CPC A61K 35/17; A61K 40/11; A61K 40/31; A61K 40/4257; A61P 35/00; C07K 14/7051; C07K 16/3092; C12N 5/0636; A61K 2239/13; A61K 2239/54; C07K 2317/622

(57) ABSTRACT

This disclosure relates to compositions and methods of treating disorders such as cancer using immune effector cells (e.g., T cells or NK cells) that express a chimeric antigen receptor (CAR). In certain embodiment, this disclosure relates to methods of using a CAR-expressing cell therapy in combination with a vasoactive intestinal peptide receptor antagonist.

### CROSS-REFERENCE TO RELATED APPLICATIONS

This application claims the benefit of U.S. Provisional Application No. 63/264,823 filed Dec. 2, 2021. The entirety of this application is hereby incorporated by reference for all purposes.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22017PCT.xml. The XML file is 26 KB, was created on Dec. 1, 2022, and is being submitted electronically via the USPTO patent electronic filing system.

### BACKGROUND

To improve the ability of immune cells to kill cancerous cells, T cells can be isolated from the blood of a patient and altered to express chimeric antigen receptors (CARs) that specifically target proteins expressed on the surface of cancerous cells and stimulate an immune response. When put back into the patient, the cells attack the cancerous cells. Brentjens et al. report that T cells altered to bind CD19 can induce remissions of cancer in adults with chemotherapy-refractory acute lymphoblastic leukemia. Several CAR base therapies are clinically approved for treating certain hematological cancers. However, CAR therapies are reported to be less effective at treating solid tumors. Thus, there is a need to identify improvements.

Vasoactive intestinal peptide (VIP) is neuropeptide with immunosuppressive effects on T cells. Li et al. report modulation of immune checkpoints and graft-versus-leukemia in allogeneic transplants by antagonizing vasoactive intestinal peptide signaling. Cancer Res, 2016, 76(23):6802-6815.

Petersen report that administration of a vasoactive intestinal peptide antagonist enhances the autologous anti-leukemia T cell response in murine models of acute leukemia. Oncoimmunology, 2017, 6(5) e1304336.

Waller et al. report VIP and VIP agonists, nanoparticles, and uses in inflammatory T-cell mediated disease. WO 2019/240881. References reported herein are not an admission of prior art.

### SUMMARY

This disclosure relates to compositions and methods of treating disorders such as cancer using immune effector cells (e.g., T cells or NK cells) that express a chimeric antigen receptor (CAR). In certain embodiment, this disclosure relates to methods of using a CAR-expressing cell therapy in combination with a vasoactive intestinal peptide receptor antagonist.

In certain embodiments, this disclosure relates to compositions comprising a CAR co-expressing a vasoactive intestinal peptide receptor antagonist and uses thereof. In some embodiments, the CAR molecule and the vasoactive intestinal peptide receptor antagonist are encoded by a single nucleic acid molecule. In some embodiments, the CAR molecule and the vasoactive intestinal peptide receptor antagonist are encoded by separate nucleic acid molecules.

In certain embodiments, it is contemplated that a cell co-expressing a CAR and the vasoactive intestinal peptide receptor antagonist has enhanced expansion and proliferative capacity. In some embodiments, a cell co-expressing a CAR and the vasoactive intestinal peptide receptor antagonist has improved efficacy.

In certain embodiments, this disclosure relates to nucleic acids, e.g., a recombinantly produced nucleic acid molecule, comprising: a first nucleic acid sequence encoding a chimeric antigen receptor (CAR) that binds to an antigen (e.g., CD19, Muc16 or C-terminal fragment thereof) and a second nucleic acid sequence encoding a vasoactive intestinal peptide receptor antagonist. In certain embodiments, the first nucleic acid sequence and second nucleic acid sequence are disposed on a single nucleic acid molecule, e.g., a vector, a viral vector, retroviral vector, or a lentiviral vector. In certain embodiments, the first nucleic acid sequence and second nucleic acid sequence are disposed on separate nucleic acid molecules, e.g., separate vectors, separate viral vectors, separate retroviral vectors, separate lentivirus vectors or combinations thereof.

In certain embodiments, the single nucleic acid molecule has the following arrangement encoding a peptide in an N-terminal to C-terminal orientation: the first nucleic acid sequence, a linker, the second nucleic acid sequence wherein the linker encodes a self-cleavage site, such as e.g., a P2A site. In certain embodiments, the first nucleic acid sequence encodes a chimeric antigen. In certain embodiments, the second nucleic acid sequence encodes the vasoactive intestinal peptide receptor antagonist.

In certain embodiments, the vasoactive intestinal peptide receptor antagonist is a hybrid peptide of neurotensin and a VIP Receptor antagonist consisting of an N-terminal KPRRPY (SEQ ID NO: 2).

In certain embodiments, the vasoactive intestinal peptide receptor antagonist is

(SEQ ID NO: 1)
KPRRPYTDNYTRLRKQMAVKKYLNSILN (VIP-hyb).

In certain embodiments, the vasoactive intestinal peptide receptor antagonist has of the amino acid of K P R R P Y X¹ X² N X³ T X⁴ L R K Q X⁵ A V X⁶ K Y X⁷ N X⁸ I L N (SEQ ID NO: 12), wherein X¹ is A or any amino acid; X² is V or any amino acid; X³ is C or any amino acid; X⁴ is S or any amino acid; X⁵ is I or any amino acid; X⁶ is N or any amino acid; X⁷ is M or any amino acid; X⁸ is I or any amino acid.

In certain embodiments, the vasoactive intestinal peptide receptor antagonist has of the amino acid sequence of

(SEQ ID NO: 3)
KPRRPYADNYTRLRKQMAVKKYLNSILN (Ant-001),
(SEQ ID NO: 4)
KPRRPYTVNYTRLRKQMAVKKYLNSILN (Ant-002),
(SEQ ID NO: 5)
KPRRPYTDNCTRLRKQMAVKKYLNSILN (Ant-003),
(SEQ ID NO: 6)
KPRRPYTDNYTSLRKQMAVKKYLNSILN (Ant-004),
(SEQ ID NO: 7)
KPRRPYTDNYTRLRKQIAVKKYLNSILN (Ant-05),
(SEQ ID NO: 8)
KPRRPYTDNYTRLRKQMAVNKYLNSILN (Ant-06),
(SEQ ID NO: 9)
KPRRPYTDNYTRLRKQMAVKKYMNSILN (Ant-07),
or
(SEQ ID NO: 10)
KPRRPYTDNYTRLRKQMAVKKYLNLILN (Ant-08).

In certain embodiments, the vasoactive intestinal peptide receptor antagonist has of the amino acid sequence of KPRRPYTSDYTRLRKQMAVKKYLNLILN (Ant-308) (SEQ ID NO: 12).

In certain embodiments, this disclosure relates to recombinant vectors comprising a nucleic acid disclosed herein in operable combination with a heterologous promotor.

In certain embodiments, the recombinant vector is a viral vector, retroviral vector, or lentiviral vector.

In certain embodiments, this disclosure relates to immune cells comprising a nucleic acid or recombinant vector as disclosed herein.

In certain embodiments, this disclosure relates to a population of immune cells as disclosed herein of which more than 15%, 25%, 35%, or 50% of the immune cells comprise a nucleic acid or recombinant vector or CAR expressed therefrom as disclosed herein.

In certain embodiments, the immune cell or population of immune cells are selected from T cells or natural killer cells.

In certain embodiments, this disclosure relates to methods of treating cancer or other immune cell disease or condition comprising, contacting, inserting, or transfecting the immune cells with a nucleic acid encoding a chimeric antigen receptor and a nucleic acid encoding a vasoactive intestinal peptide receptor antagonist; and administering an effective amount of a chimeric antigen receptor expressing immune cells and a vasoactive intestinal peptide receptor antagonist expressing immune cells to a subject in need thereof.

In certain embodiments, the subject is diagnosed with elevated blood serum levels of CA-125 (N-terminal fragment of Muc16). In certain embodiments, a sample from the subject contains elevated levels of CA-125 when compared to a normal or reference value.

In certain embodiments, immune cells expressing the chimeric antigen receptor and vasoactive intestinal peptide receptor antagonist, prior to administration, are contacted with beads or other solid surfaces comprising an antibodies or other specific binding agents that specifically bind CD3 and CD28 providing activate immune cells.

In certain embodiments, the immune cells expressing chimeric antigen receptor and vasoactive intestinal peptide receptor antagonist are, prior to administration, contacted with IL-2 providing activate immune cells.

In certain embodiments, isolating immune cells is from the subject diagnosed with cancer or other immune cell disease or condition (autologous). In certain embodiments, isolating immune cells is not from the subject diagnosed with cancer or other immune cell disease or condition (allogeneic or syngeneic).

In certain embodiments, the cancer is a hematological cancer, solid tissue cancer, tumor, metastatic cancer, or other neoplasm.

In certain embodiments, administering is in combination with administering an additional anticancer agent or active agent.

In certain embodiments, this disclosure relates to compositions comprising the cells disclosed herein. In certain embodiments, the composition is a pharmaceutical composition further comprising a pharmaceutically acceptable carrier.

### BRIEF DESCRIPTION OF THE SEVERAL VIEWS OF THE DRAWINGS

FIG. 1 illustrates a pancreatic ductal adenocarcinoma (PDAC) Muc-16 targeted CAR T cell therapy for overcome immunosuppressive mechanisms of the tumor microenvironment by co-expression of a vasoactive intestinal peptide receptor antagonist.

FIGS. 2A-2C show data indicating antigen-stimulation increases VPAC1+ CAR T cells and VIPR antagonism enhances CAR T cell proliferation in the presence of VIP.

FIG. 2A shows data where CAR T cells were co-cultured alone or with antigen-relevant or antigen-irrelevant CAR T cells for 24 hrs. VPAC1 and VPAC2 expression was evaluated via flow cytometry.

FIG. 2B shows data indicating antigen-stimulated CAR T cells express significantly more VIP.

FIG. 2C shows data where CAR T cells co-cultured with antigen-relevant target cells were treated with exogenous VIP and/or VIPR antagonist indicating the proliferation of CAR T cells is suppressed by VIP and rescued by exogenous VIPR antagonist.

FIG. 3 shows data indicating patient-derived xenograft (PDX) tumors expressing VIP and Muc16-CD can be lysed by anti-Muc16-CD CAR T in vitro. Cleavage of Muc16 produces serum biomarker CA125 and the retained oncogenic ectodomain Muc16-CD. The three-dimensional retained ectodomain of Muc16 (Muc16-CD) can be detected on the surface of in PDX tumor cell lines by flow cytometry. VIP is expressed by a human pancreatic cell line (Panc1) and PDX tumor cell lines. Data indicates that anti-Muc16-CD CAR T cells are capable of cytotoxic function against Muc16-CD-expressing PDX tumor cells lines.

FIG. 4A illustrates constructs encoding Muc16-targeted VIPR antagonist-secreting CAR. Transduction of human primary T cells with anti-Muc16CD-antVIPR CAR provided VIPR antagonist-secreting CAR T cells.

FIG. 4B shows data indicating the anti-Muc16CD-antVIPR CARs are capable of cytotoxic function and cytokine production.

FIG. 5A shows data indicating CAR T cell-producing VIPR antagonist peptides (AntVIPR-CAR T cells) have reduced VIP.

FIG. 5B shows data indicating antVIPR CAR T cells have increased activation (CD69+) after antigen-stimulation.

FIG. 5C shows data indicating AntVIPR CAR T cells confer a proliferative advantage when expanded in vitro compared to parental CAR T cells.

FIG. 6A illustrates a treatment schedule and experimental conditions wherein SCID/Beige mice were engrafted with Panc1-Muc16CD tumors and treated with CAR T cells 5-7 days later intraperitoneally.

FIG. 6B shows a Kaplan-Meier survival curve with data from mice treated with anti-Muc16CD CAR T and anti-Muc16CD-antVIPR CAR T cells. Human T cells were detectable in anti-Muc16CD-antVIPR CAR T treated mice on days 118 and 204.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. Thus, reference to an “embodiment” refers to an example of the invention and is not necessarily limited by such an example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, physiology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “vasoactive intestinal peptide” and “VIP” refer a multifunctional endogenous polypeptide that modulates innate and adaptive immunity. See VIP peptides isoform X1 [Homo sapiens] NCBI Reference Sequence No. XP_006715625.1 and hormone domain 2. VIP is typically secreted by a variety of cells such as neurons (in both the central and peripheral nervous systems) B-cells, T-cells, and accessory cells. VIP and the closely related neuropeptide pituitary adenylyl cyclase-activating polypeptide (PACAP) bind to three known receptors—VPAC1, VPAC2, and PAC1. It is believed that T-cells and dendritic cells (DC) express VPAC1 and VPAC2, but not PAC1. PAC1 is mainly expressed on neuron and endocrine cells in the brain and pituitary and adrenal glands, and in most forms selectively binds PACAP.

The term “VIP antagonist” or “VIP receptor antagonist” refers to any molecule that inhibits or detracts from the ability of VIP to alter immune responses. VIP receptor antagonists are known including VIP analogues, VIP fragments, growth hormone-releasing factor analogs and hybrid peptides. Numerous VIP receptor antagonists are disclosed in U.S. Pat. Nos. 5,565,424; 7,094,755; 6,828,304, and PCT International Publication Number WO 2020/102694 all are hereby incorporated by reference.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells). The neoplasia can be a primary tumor or primary cancer. In addition, the neoplasm can be in metastatic status.

“Subject” refers any animal, preferably a human patient, livestock, or domestic pet.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse, or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells and/or agents that are administered.

As used herein, the term “combination with” when used to describe administration with an additional treatment means that the agent may be administered prior to, together with, or after the additional treatment, or a combination thereof so long as the agent are present in the patient/subject at as some overlapping time.

The term “specific binding agent” refers to a molecule, such as a proteinaceous molecule, that binds a target molecule with a greater affinity than other random molecules or proteins. Examples of specific binding agents include an antibody that bind an epitope of an antigen or a receptor which binds a ligand. In certain embodiments, “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen, and thereby targeting the antigen for degradation, or any organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibodies, or non-naturally produced synthetic antibodies, such as specific binding single chain antibodies, bispecific antibodies, or fragments thereof. These antibodies may have chemical modifications.

From a structural standpoint, a natural human antibody is a combination of proteins: two heavy chain proteins and two light chain proteins. The heavy chains are longer than the light chains. The two heavy chains typically have the same amino acid sequence. Similarly, the two light chains typically have the same amino acid sequence. Each of the heavy and light chains contain a variable segment that contains amino acid sequences which typically participate in binding to the antigen. The variable segments of the heavy chain do not have the same amino acid sequences as the light chains. The variable segments are often referred to as the antigen binding domains. Recombinantly produced single chains of the variable regions of the heavy chain and/or light chain are often alone sufficient for specific binding to an antigen, although usually to a lesser extent than the complete antibody. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other expression system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “heterologous” nucleic acid sequence or peptide sequence refers to a nucleic acid sequence or a peptide sequence that does not naturally occur, e.g., because the whole sequence contains a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a peptide refers to a protein molecule that is expressed using a recombinant nucleic acid molecule.

The term “vector” refers to a recombinant nucleic acid containing a desired coding sequence and appropriate nucleic acid sequences necessary for the expression of the operably linked coding sequence in a particular host organism or expression system, e.g., cellular or cell-free expression systems. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In certain embodiments, this disclosure contemplates a vector encoding a peptide disclosed herein in operable combination with a heterologous promoter.

With regard to variants, any type of alteration in the amino acid, or nucleic acid, sequence is permissible so long as the resulting variant sequence functions for its intended purpose. Examples of such variations include, but are not limited to, deletions, insertions, substitutions, and combinations thereof. For example, with regard to proteins, it is well understood by those skilled in the art that one or more (e.g., 2, 3, 4, 5, 6, 7, 8, 9 or 10), amino acids can often be removed from the amino and/or carboxy terminal ends of a protein without significantly affecting the activity of that protein. Similarly, one or more (e.g., 2, 3, 4, 5, 6, 7, 8, 9 or 10) amino acids can often be inserted into a protein, or deleted from a protein, without significantly affecting the activity of the protein depending on the location within the protein.

With specific regard to proteins, any amino acid substitution is permissible so long as the activity of the protein is not significantly affected. In this regard, it is appreciated in the art that amino acids can be classified into groups based on their physical properties. Examples of such groups include, but are not limited to, charged amino acids, uncharged amino acids, polar uncharged amino acids, and hydrophobic amino acids. Preferred variants that contain substitutions are those in which an amino acid is substituted with an amino acid from the same group. Such substitutions are referred to as conservative substitutions. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

In certain embodiments, variants of amino acid sequences disclosed herein comprise or consists of at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence.

In certain embodiments, term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position.

### Chimeric Antigen Receptors

As used herein, a “chimeric antigen receptor” or “CAR” refers to a protein receptor, which introduces an antigen specificity (specific binding agent), via an antigen binding domain, onto cells (immune cells) to which it is expressed in immune cells (for example T cells such as naive T cells, central memory T cells, effector memory T cells or combination thereof) thus combining the antigen binding properties of the antigen binding domain with the T cell activity (e.g. lytic capacity). A CAR typically includes an extracellular antigen-binding domain (specific binding ectodomain), a transmembrane domain and an intracellular signaling domain. The intracellular signaling domain generally contains at least one immunoreceptor tyrosine-based activation motif (ITAM) signaling domain, e.g., derived from CD3zeta, and optionally at least one costimulatory signaling domain, e.g., derived from CD28 or 4-1BB.

To improve the ability of immune cells to kill cancerous cells, T cells can be isolated from the blood of a patient and modified with a recombinant vector to express chimeric antigen receptors (CARs) that specifically target proteins expressed on the surface of cancerous cells and stimulate an immune response.

In certain embodiments, the targeting sequence in a chimeric antigen receptor refers to any variety of molecules or polypeptide sequences capable of selectively binding to a targeted associated molecule. The targeting sequences may be derived from variable binding regions of antibodies, single chain antibodies, and antibody mimetics. In certain embodiments, targeting sequence is a single-chain variable fragment (scFv) derived from an antibody. The targeting sequence is typically connected to intracellular domains by a hinge/transmembrane region, commonly derived from CD8 or IgG4. The intracellular domains may contain co-stimulatory domains such as CD80, CD86, 4-1BBL, IL-2Rbeta, OX40L and CD70 and/or CD28 linked to the cytoplasmic signaling domain of CD3zeta.

Whole blood is composed of plasma, red blood cells (RBCs; or erythrocytes), platelets, and nucleated white blood cells, also referred to as leukocytes. The leukocytes can be further categorized into mononuclear cells and polymorphonuclear cells (or granulocytes). There are different techniques to obtain peripheral blood mononuclear cells (PBMCs), polymorphonuclear cells, leukocytes, or specific cell subsets, e.g., isolate specific cells directly by using flow cytometry, depleting red blood cells, centrifugation, and/or apheresis. Peripheral blood mononuclear cells (PBMCs) may be isolated by leukapheresis. T cells can be enriched by mononuclear cells counter-flow elutriation and expanded by addition of anti-CD3/CD28 antibody coated paramagnetic beads for activation of T cells. Cells may be expanded, harvested, and cryopreserved in infusible medium sometime after the subject has had an allogeneic stem-cell transplantation. Cells may be obtained by isolation from peripheral blood and optionally purified by fluorescent activated cells sorting e.g., mixing cells with fluorescent antibodies or other fluorescent agents (molecular beacons) and separating the cells by flow cytometry based fluorescent sorting.

Cluster of differentiation 3 (CD3) is expressed on T cells as it is associated with the T cells receptor (TCR). The majority of TCR are made up of alpha beta chains (alpha beta T-cells). Alpha beta T-cells typically become double-positive intermediates (CD4+CD8+) which mature into single-positive (CD4+CD8−) T helper cells or (CD4−CD8+) cytotoxic T cells. T helper cells interact with antigen presenting dendritic cells and B cells. Upon activation with cognate antigen by dendritic cells, antigen specific CD4 T cells can differentiate to become various types of CD4 T cells with specific roles in promoting immune responses. T cells may be isolated and separated from a human sample (blood or PBMCs or bone marrow) based on the expression of alpha beta T cells receptor (TCR), gamma delta T cells receptor, CD2, CD3, CD4, CD8, CD4 and CD8, NK1.1, CD4 and CD25 and other combinations based on positive or negative selection. In a typical procedure, T cells are purified and isolated from blood or bone marrow. For example, T cells are collected via apheresis, a process that withdraws blood from the body and removes one or more blood components (such as plasma, platelets, or other white blood cells). The remaining blood is then returned into the body. The cells are exposed to a recombinant vector, such as a lentiviral or retroviral vector, that infects the cells in a way that a chimeric antigen receptor (CAR) protein is produced and presented in the cell membrane.

In certain embodiments, an immune cell is selected from the group consisting of a T cell, a natural killer (NK) cell, and a stem cell from which a lymphoid cell may be differentiated. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell. In certain embodiments, the T cell is a CD4+ T cell. In certain embodiments, the T cell is a CD8+ T cell.

### CAR T Cells Expressing Vasoactive Intestinal Peptide Receptor Antagonist

In certain embodiments, this disclosure relates to compositions comprising a CAR co-expressing a vasoactive intestinal peptide receptor antagonist and uses thereof. In some embodiments, the CAR molecule and the vasoactive intestinal peptide receptor antagonist are encoded by a single nucleic acid molecule. In some embodiments, the CAR molecule and the vasoactive intestinal peptide receptor antagonist are encoded by separate nucleic acid molecules.

In certain embodiments, it is contemplated that a cell co-expressing a CAR and the vasoactive intestinal peptide receptor antagonist has enhanced expansion and proliferative capacity. In some embodiments, a cell co-expressing a CAR and the vasoactive intestinal peptide receptor antagonist has improved efficacy.

In certain embodiments, this disclosure relates to nucleic acids, e.g., a recombinantly produced nucleic acid molecule, comprising: a first nucleic acid sequence encoding a chimeric antigen receptor (CAR) that binds to an antigen (e.g., an antigen described herein, e.g., Muc16 (carboxy terminal domain or nitrogen terminal domain) or CD19; a second nucleic acid sequence encoding a vasoactive intestinal peptide receptor antagonist. In certain embodiments, the first nucleic acid sequence and second nucleic acid sequence are disposed on a single nucleic acid molecule, e.g., a vector, a viral vector, or a lentivirus vector. In certain embodiments, the first nucleic acid sequence and second nucleic acid sequence are disposed on separate nucleic acid molecules, e.g., separate vectors, separate viral vectors, separate retroviral vectors, separate lentivirus vectors, or combinations thereof.

In certain embodiments, the single nucleic acid molecule encodes one or more proteins having the following arrangement in an N-terminal to C-terminal orientation: the first nucleic acid sequence, a linker, the second nucleic acid sequence wherein the linker encodes a self-cleavage site, such as e.g., a P2A site, a T2A site, an E2A site, or an F2A site. In certain embodiments, the first nucleic acid sequence encodes a chimeric antigen. In certain embodiments, the second nucleic acid sequence encodes the vasoactive intestinal peptide receptor antagonist.

In certain embodiments, the vasoactive intestinal peptide receptor antagonist is a hybrid peptide of neurotensin and a VIP Receptor antagonist consisting of an N-terminal KPRRPY (SEQ ID NO:2).

In certain embodiments, the vasoactive intestinal peptide receptor antagonist is

(SEQ ID NO: 1)
KPRRPYTDNYTRLRKQMAVKKYLNSILN (VIP-hyb).

In certain embodiments, the vasoactive intestinal peptide receptor antagonist has of the amino acid of K P R R P Y X¹ X² N X³ T X⁴ L R K Q X⁵ A V X⁶ K Y X⁷ N X⁸ I L N (SEQ ID NO: 11), wherein X¹ is A or any amino acid; X² is V or any amino acid; X³ is C or any amino acid; X⁴ is S or any amino acid; X⁵ is I or any amino acid; X⁶ is N or any amino acid; X⁷ is M or any amino acid; X⁸ is I or any amino acid.

In certain embodiments, the vasoactive intestinal peptide receptor antagonist has of the amino acid of

(SEQ ID NO: 3)
KPRRPYADNYTRLRKQMAVKKYLNSILN (Ant-001),
(SEQ ID NO: 4)
KPRRPYTVNYTRLRKQMAVKKYLNSILN (Ant-002),
(SEQ ID NO: 5)
KPRRPYTDNCTRLRKQMAVKKYLNSILN (Ant-003),
(SEQ ID NO: 6)
KPRRPYTDNYTSLRKQMAVKKYLNSILN (Ant-004),
(SEQ ID NO: 7)
KPRRPYTDNYTRLRKQIAVKKYLNSILN (Ant-05),
(SEQ ID NO: 8)
KPRRPYTDNYTRLRKQMAVNKYLNSILN (Ant-06),
(SEQ ID NO: 9)
KPRRPYTDNYTRLRKQMAVKKYMNSILN (Ant-07),
or
(SEQ ID NO: 10)
KPRRPYTDNYTRLRKQMAVKKYLNLILN (Ant-08).

In certain embodiments, the vasoactive intestinal peptide receptor antagonist has of the amino acid of KPRRPYTSDYTRLRKQMAVKKYLNLILN (Ant-308) (SEQ ID NO: 12).

In certain embodiments, a single nucleic acid as reported herein may encode at least one cytokine or a fragment thereof. In certain embodiments, the cytokine is selected from the group consisting of IL-18, IL-36, IL-33, IL-12, IL-21, and IL-2.

In certain embodiments, this disclosure relates to recombinant vectors comprising a nucleic acid disclosed herein in operable combination with a heterologous/exogenous promotor.

In certain embodiments, the nucleic acid composition further comprises a heterologous promoter that is operably linked to express a protein having the chimeric antigen receptor and vasoactive intestinal peptide receptor antagonist linked with a self-cleaving peptide.

In certain embodiments, the nucleic acid composition further comprises a first heterologous promoter that is operably linked to express a protein having the chimeric antigen receptor and a second heterologous promotor operably linked to express a vasoactive intestinal peptide receptor antagonist. In certain embodiments, the first and second heterologous promoter may the same or different.

In certain embodiments, the heterologous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and gamma retrovirus 5′ long-terminal repeat (LTR) promoter. In certain embodiments, the promoter is an inducible promoter. In certain embodiment, the inducible promoter is selected from the group consisting of a NFAT transcriptional response element (TRE) promoter, a CD69 promoter, a CD25 promoter, Nur77 promoter, and an IL-2 promoter. In certain embodiments, the promoter is endogenous.

In certain embodiments, the recombinant vector is a viral vector, retroviral vector, or lentiviral vector.

In certain embodiments, the chimeric antigen receptor and a vasoactive intestinal peptide receptor antagonist can be constructed in a single, polycistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, the polycistronic expression cassette comprises a P2A self-cleaving peptide.

In certain embodiments, the extracellular antigen-binding domain of the chimeric antigen receptor is a single-chain variable fragment (scFv), a Fab, or a F(ab)2. In certain embodiments, one or more of the scFv, Fab and F(ab)2 are comprised in a fusion protein with a heterologous sequence to form the extracellular antigen-binding domain. In certain embodiments, the extracellular antigen-binding domain is a humanized scFv.

In certain embodiments, the extracellular antigen-binding domain binds Muc16, Muc16 carboxy terminal domain, or Muc16 amino terminal domain. In certain embodiments, the CAR antigen-binding domain Muc16 carboxy terminal domain comprises a heavy chain variable region and/or a light chain variable region, wherein
a) the heavy chain variable region comprises a CDR1 comprising the amino acid sequence SYAMS (SEQ ID NO: 13) or a conservative modification thereof, a CDR2 comprising the amino acid sequence TISSAGGYIFYSDSVQG (SEQ ID NO: 14) or a conservative modification thereof, and a CDR3 comprising the amino acid sequence QGFGNYGDYYAMDY (SEQ ID NO: 15) or a conservative modification thereof, and/or
b) the light chain variable region comprises a CDR1 comprising the amino acid sequence KSSQSLLNSRTRKNQLA (SEQ ID NO: 16) or a conservative modification thereof, a CDR2 comprising the amino acid sequence WASTRQS (SEQ ID NO: 17) or a conservative modification thereof, and a CDR3 comprising the amino acid sequence QQSYNLLT (SEQ ID NO: 18) or a conservative modification thereof.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence
EVKLQESGGGFVKPGGSLKVSCAASGFTF SSYAMSWVRLSPEMRLEWVATISSA GGYIFYSDSVQGRFTISRDNAKNTLHLQMGSLRSGDTAMYYCARQGFGNYGDYYAMD YWGQGTTVTVSS (SEQ ID NO: 19). In certain embodiments, the heavy chain variable region comprises the amino acid sequence set forth in SEQ ID NO: 19.

In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence
IELTQSPSSLAVSAGEKVTMSCKSSQSLLNSRTRKNQLAWYQQKPGQSPELLIYW ASTRQSGVPDRFTGSGSGTDFTLTISSVQAEDLAVYYCQQSYNLLTFGPGTKLEIKR (SEQ ID NO: 20). In certain embodiments, the light chain variable region comprises the amino acid sequence set forth in SEQ ID NO: 20.

In certain embodiments, the heavy chain variable region comprises the amino acid sequence set forth in SEQ ID NO: 19; and the light chain variable region comprises the amino acid sequence set forth in SEQ ID NO: 20.

In certain embodiments, the extracellular antigen-binding domain binds CD19. In certain embodiments, the CAR antigen-binding domain CD19 comprises a heavy chain variable region and/or a light chain variable region, wherein
a) the heavy chain variable region comprises a CDR1 comprising the amino acid sequence SYWMN (SEQ ID NO: 21) or a conservative modification thereof, a CDR2 comprising the amino acid sequence QIYPGDGDTNYNGKFKG (SEQ ID NO: 22) or a conservative modification thereof, and a CDR3 comprising the amino acid sequence KTISSVVDFYFDY (SEQ ID NO: 23) or a conservative modification thereof, and/or
b) the light chain variable region comprises a CDR1 comprising the amino acid sequence KASQNVGTNVA (SEQ ID NO: 24) or a conservative modification thereof, a CDR2 comprising the amino acid sequence SATYRNS (SEQ ID NO: 25) or a conservative modification thereof, and a CDR3 comprising the amino acid sequence QQYNRYPYT (SEQ ID NO: 26) or a conservative modification thereof.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence
EVKLQQSGAELVRPGSSVKISCKASGYAFSSYWMNWVKQRPGQGLEWIGQIYP GDGDTNYNGKFKGQATLTADKSSSTAYMQLSGLTSEDSAVYFCARKTISSVVDFYFDY WGQGTTVTVSS (SEQ ID NO: 27). In certain embodiments, the heavy chain variable region comprises the amino acid sequence set forth in SEQ ID NO: 27.

In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence
DIELTQSPKFMSTSVGDRVSVTCKASQNVGTNVAWYQQKPGQSPKPLIYSATYR NSGVPDRFTGSGSGTDFTLTITNVQSKDLADYFCQQYNRYPYTSGGGTKLEIKR (SEQ ID NO: 28). In certain embodiments, the light chain variable region comprises the amino acid sequence set forth in SEQ ID NO: 28.

In certain embodiments, the heavy chain variable region comprises the amino acid sequence set forth in SEQ ID NO: 27; and the light chain variable region comprises the amino acid sequence set forth in SEQ ID NO: 28.

In certain embodiments, the transmembrane domain comprises a CD8 polypeptide, a CD28 polypeptide, a CD3zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof. In certain embodiments, the transmembrane domain comprises a CD28 polypeptide.

In certain embodiments, the intracellular signaling domain comprises a CD3zeta polypeptide. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide or a 4-1BB polypeptide. In certain embodiments, the at least one co-stimulatory signaling region comprises a CD28 polypeptide.

### CAR T Cells Expressing Vasoactive Intestinal Peptide Receptor Antagonists for Uses in Treating Cancers

This disclosure relates to compositions and methods of treating disorders such as cancer using immune effector cells (e.g., T cells or NK cells) that express a chimeric antigen receptor (CAR), e.g., a CAR that binds to a tumor antigen or an antigen expressed on the surface of a solid tumor or a hematological tumor. In certain embodiment, this disclosure relates to methods of using a CAR-expressing cell therapy in combination with a vasoactive intestinal peptide receptor antagonist.

In certain embodiments, this disclosure relates to immune cells comprising a nucleic acid or recombinant vector as disclosed herein.

In certain embodiments, this disclosure relates to a population of immune cells as disclosed herein of which more than 15%, 25%, 35%, or 50% of the immune cells comprise a nucleic acid or recombinant vector as disclosed herein.

In certain embodiments, the immune cell is a cell of the lymphoid lineage or a cell of the myeloid lineage. In certain embodiments, the immune cell is selected from the group consisting of a T cell, a Natural Killer (NK) cell, and a stem cell from which a lymphoid cell may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell. In certain embodiments, the T cell is a CD4+ T cell. In certain embodiments, the T cell is a CD8+ T cell. The cells (e.g., T cells) can be autologous, non-autologous (e.g., allogeneic), or derived in vitro from engineered progenitor or stem cells.

In certain embodiments, this disclosure relates to methods of treating cancer or other immune cell disease or condition comprising, contacting, inserting, or transfecting the immune cells with a peptide having or nucleic acid encoding a chimeric antigen receptor and a nucleic acid encoding a vasoactive intestinal peptide receptor antagonist; and administering an effective amount of a chimeric antigen receptor expressing and a vasoactive intestinal peptide receptor antagonist expressing immune cells to a subject in need thereof.

In certain embodiments, the subject is diagnosed with elevated blood serum levels of CA-125 (N-terminal Muc16). In certain embodiments, a sample from the subject is contains elevated levels of CA-125 when compared to a normal or reference value.

In certain embodiments, chimeric antigen receptor expressing and vasoactive intestinal peptide receptor antagonist expressing immune cells, prior to administration, are contacted with beads or other solid surfaces comprising an antibodies or other specific binding agents that specifically bind CD3 and CD28 providing activate immune cells.

In certain embodiments, chimeric antigen receptor expressing and vasoactive intestinal peptide receptor antagonist expressing immune cells are, prior to administration, contacted with a cytokine and/or interleukin, e.g., IL-2, providing activate immune cells.

In certain embodiments, isolating immune cells is from the subject diagnosed with cancer or other immune cell disease or condition (autologous). In certain embodiments, isolating immune cells is not from the subject diagnosed with cancer or other immune cell disease or condition (allogeneic or syngeneic).

In certain embodiments, the cancer is a hematological cancer, solid tissue cancer, tumor, or metastatic cancer.

In certain embodiments, this disclosure relates to methods of reducing tumor burden in a subject. In certain embodiments, the method comprises administering an effective amount of presently disclosed cells or composition to the subject. In certain embodiments, the method reduces the number of tumor cells, reduces tumor size, and/or eradicates the tumor in the subject. The presently disclosed subject matter further provides methods of increasing or lengthening survival of a subject having a tumor or neoplasm. In certain embodiments, the method comprises administering an effective amount of presently disclosed cells or composition to the subject. The presently disclosed subject matter further provides methods of treating and/or preventing a tumor or neoplasm in a subject.

In certain embodiments, the cells express a chimeric antigen receptor and optionally macrophage colony-stimulating factor 1 receptor (CSF-1R).

In certain embodiments, the activated cells are administered is in combination with an additional chemotherapy agent. In certain embodiments, the additional chemotherapy agent is a checkpoint inhibitor.

In certain embodiments, administering is implanting, injecting the activated cells in proximity of a tumor or tissue comprising cancerous cells, e.g., inside or within 1, 2, or 3 cm of a solid tumor mass or tissue containing cancerous cells.

In certain embodiments, the cells are infused directly to tumors at various anatomical sites, such as the brain, breast, thorax, lung, and liver.

In certain embodiments, the cells are unmodified or genetically engineered. In certain embodiments, the immune cells express a chimeric antigen receptor as reported herein. In certain embodiments, the recombinant gene construct is a recombinant vector, virus, lentivirus, or retrovirus, and virally or non-virally introduced into immune cells, e.g., introduced into immune cells with a retrovirus, introduced into immune cells with transposon-based genomic integration, introduced into immune cells with electroporation, introduced into immune cells with mechanoporation.

In certain embodiments, the antigen binding domain or the chimeric antigen receptor specifically binds (MUC16) mucin16 (N-terminal or C-terminal), (MUC1) mucin1, (EGFR) epidermal growth factor receptor, (HER2) human epidermal growth factor receptor 2, (EpCAM) epithelial cell adhesion molecule, (AFP) alpha-fetoprotein, (FAP) familial adenomatous polyposis, (CEA) carcinoembryonic antigen, (PSCA) prostate stem cell antigen, (PSMA) prostate-specific membrane antigen, (PSA) prostate-specific antigen, (AXL) AXL receptor tyrosine kinase, (DLL3) delta-like 3, (EPHA2) EPH receptor A2, (FRα) folate receptor alpha, (LMP1) Epstein-Barr virus latent membrane protein 1, (MAGE) melanoma antigen gene protein, MAGE-A1, MAGE-A3, MAGE-A4, (DR5) death receptor 5, (NKG2D) natural killer group 2 member D receptor, (CAIX) carbonic anhydrase IX, (TAG-72) tumor-associated glycoprotein 72, (GUCY2C) guanylate cyclase 2C, (ANTXR1) anthrax toxin receptor 1, (GSPG4) general secretion pathway protein G, (ROR) RAR-related orphan receptors, IL13RA2 (Interleukin 13 Receptor Subunit Alpha 2), Wilms' tumor 1 (WT1), Survivin, Tn (aGalNAc-O-Ser/Thr), sialyl-Tn (aNeuAc2,6-aGalNAc-O-Ser/Thr), TF (bGal1,3-aGalNAc-O-Ser/Thr), CA 19-9 (Neu5Acα2-3Galβ1-3[Fucα1-4]GlcNAcβ), Telomerase reverse transcriptase (TERT), Beta-hCG (Human chorionic gonadotropin), p53, Ras, bladder tumor antigen (BTA), antibody specific antigen Om5, GD2 (Ganglioside GD2), integrin alpha-v/beta-6, or mesothelin antigen. In certain embodiments, the chimeric antigen receptor is an antibody single-chain variable fragment (scFv).

In certain embodiments, immune cells or T cells produced by methods disclosed herein are used in methods for treating a subject diagnosed with cancer. In certain embodiments, the cancer is a hematological malignancy such as acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), chronic myeloid leukemia (CML), myeloproliferative neoplasms (MPNs), and lymphomas, Hodgkin's lymphomas, and non-Hodgkin's lymphomas such as Burkitt lymphoma, B-cell lymphoma.

In certain embodiments, the cancer is a solid tumor, cellular malignancy, or hematological malignancy. In certain embodiments, the cancer is lung cancer, non-small cell lung cancer, small cell lung cancer, bronchus cancer, mesothelioma, malignant pleural mesothelioma, lung adenocarcinoma, breast cancer, prostate cancer, colon cancer, rectum cancer, colorectal cancer, gastrointestinal cancer, stomach cancer, esophageal cancer, ovarian cancer, cervical cancer, melanoma, kidney cancer, pancreatic cancer, pancreatic ductal adenocarcinoma (PDA), thyroid cancer, brain cancer, glioblastoma (GBM), medulloblastoma, glioma, neuroblastoma, liver cancer, bladder cancer, uterine cancer, bone cancer, osteosarcoma, sarcoma, rhabdomyosarcoma, Ewing's sarcoma, retinoblastoma, nasopharyngeal carcinoma.

In certain embodiments, the immune cells or T cells made by methods disclosed herein are used for treating cancer and are administered in combination with another anticancer agent. In certain embodiments, the anticancer agent is abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC). In certain embodiments, the anticancer agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

In certain embodiments, the cells are administered to a subject with a lymphodepleted environment due to prior or concurrent administration of lymphodepleting agents such as cyclophosphamide and fludarabine).

### Compositions and Kits

In certain embodiments, this disclosure relates to compositions and kits comprising peptides, nucleic acids, recombinant vectors, and/or cells containing the same as reported herein. In certain embodiments, this disclosure relates to compositions provided as sterile liquid preparations, e.g., isotonic aqueous solutions, suspensions, emulsions, dispersions, or viscous compositions, which may be buffered to a selected pH, e.g., between 6-8. Viscous compositions can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating a peptides, nucleic acid, vector, or cells into immune cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. In certain embodiments, a vehicle, diluent, or additive used would have to be compatible.

In certain embodiments, compositions can be isotonic, i.e., they can have the same osmotic pressure as blood. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions. Viscosity of the compositions, if desired, can be maintained at the selected level using a pharmaceutically acceptable thickening agent, e.g., methylcellulose, xanthan gum, carboxymethyl cellulose, hydroxypropyl cellulose, carbomer, and the like.

Compositions may be administered systemically or directly to a subject. In certain embodiments, the compositions are directly injected into an organ of interest (e.g., an organ affected by a neoplasia). Alternatively, the presently disclosed compositions are provided indirectly to the organ of interest, for example, by administration into the circulatory system (e.g., the tumor vasculature). Expansion and differentiation agents can be provided prior to, during or after administration to increase production of cells (e.g., T cells or NK cells) in vitro or in vivo.

In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. The cells can be introduced by injection, catheter, or the like.

In certain embodiments, cells and compositions can be administered by any method known in the art including, but not limited to, oral administration, intravenous administration, intravascular administration, intracranial administration, intraocular administration, subcutaneous administration, intranodal administration, intratumoral administration, intrathecal administration, intrapleural administration, intraosseous administration, intraperitoneal administration, pleural administration, and direct administration.

In certain embodiments, this disclosure relates to kits comprising peptides, nucleic acids, vectors, or cells disclosed herein. The kits may contain a transfer device such a needle, syringe, cannula, capillary tube, pipette, or pipette tip. In certain embodiments, the peptides, nucleic acids, or vectors may be contained in a storage container, sealed or unsealed, such a vial, bottle, blister pack, ampule, or box. In certain embodiments, the kit further comprises written instructions for using the peptides, nucleic acids, recombinant vectors, or cells as reported herein, e.g., for infecting cells, treating cancer, reducing tumor burden in a subject, treating and/or preventing a tumor or neoplasm in a subject, and/or increasing or lengthening survival of a subject having cancer, tumor, or neoplasm.

### Examples

### Engineering Optimal CAR T Cells to Secrete Vasoactive Intestinal Peptide Receptor Antagonist to Treat Pancreatic Tumors

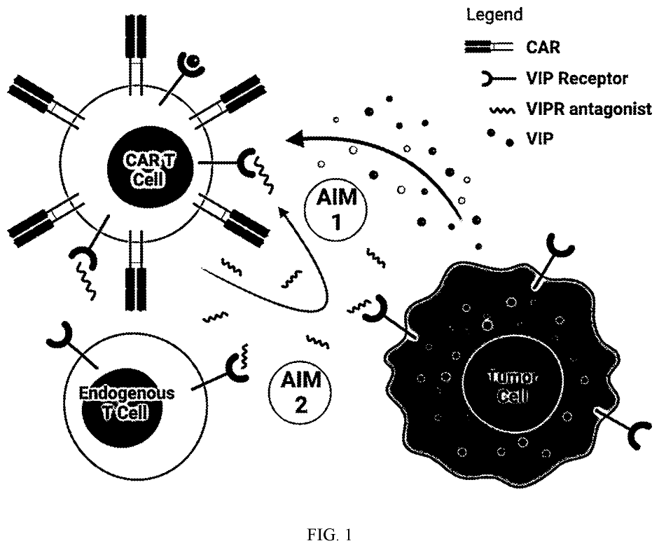

Chimeric antigen receptor (CAR) T cell therapies have shown promising clinical efficacy in hematologic cancers; however, treatment of solid tumors present challenges. Pancreatic ductal adenocarcinoma (PDAC) has a low survival rate. PDAC largely remains resistant to current immunotherapies. Reasons for this are thought to include an immunosuppressive tumor microenvironment (TME). Thus, in certain embodiments, one objective of this disclosure is to provide enhancements to CAR T cell therapy that can overcome immunosuppressive mechanisms of the TME. VIP is an immunosuppressive neuropeptide abundant in the PDAC TME. It is believed that VIPR antagonists bind to VIP receptors in competition with endogenous VIP. Data indicates that systemic VIPR antagonism (antVIPR) increases endogenous T cell activation and infiltration into the TME in a pancreatic mouse model (FIG. 1).

### VIP Alters CAR T Cell Phenotype and Function

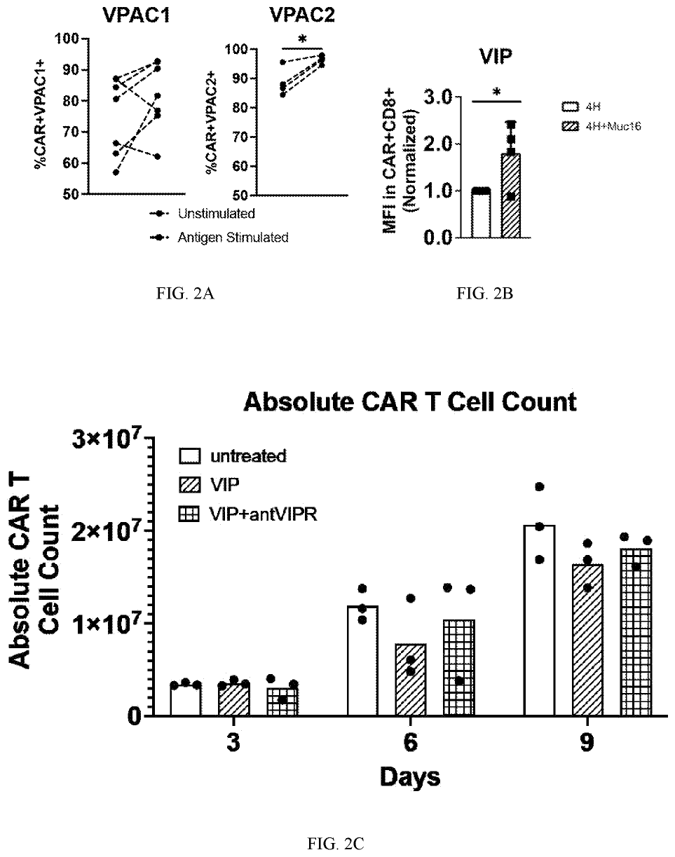

Experiments were performed to determine whether antigen-stimulation increases VPAC1+ CAR T cells and VIPR antagonism enhances CAR T cell proliferation in the presence of VIP. CAR T cells were co-cultured alone or with antigen-relevant or antigen-irrelevant CAR T cells for 24 hrs. VPAC1 and VPAC2 expression was evaluated via flow cytometry. Antigen-stimulated CAR T cells express significantly more VIP. CAR T cells co-cultured with antigen-relevant target cells were treated with exogenous VIP and/or VIPR antagonist. Proliferation of CAR T cells is suppressed by VIP and rescued by exogenous VIPR antagonist (FIGS. 2A-2C).

### Patient-Derived Pancreatic Cancer Models Express VIP and Muc16-CD

MUC16 (CA125) is a transmembrane glycoprotein overexpressed in multiple cancers including pancreatic cancer. A truncated carboxyl-terminal domain MUC16 (Muc16-CD) is generated following its cleavage releasing the N-terminal fragment (CA125) that can be detected as elevated in blood serum. Aithal et al. report the development and characterization of MUC16 carboxy-terminus specific monoclonal antibodies. PLoS ONE, 2018, 13(4): e0193907.

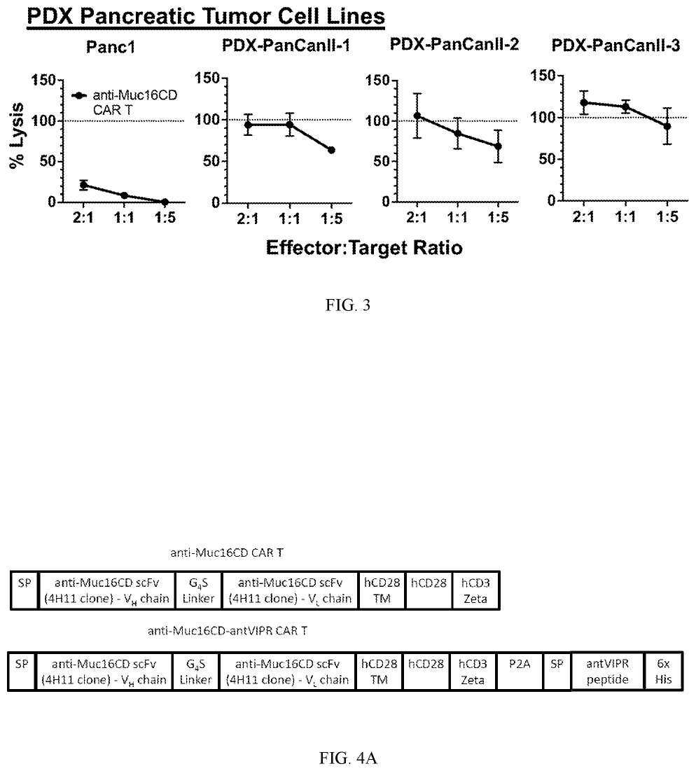

Experiments were performed to determine whether patient-derived xenograft (PDX) tumors expressing VIP and Muc16-CD can be lysed by anti-Muc16-CD CAR T in vitro. Immunofluorescent Muc16-CD staining of PDX pancreatic tumor tissues reveled that ectodomain of Muc16 (Muc16-CD) can also be detected on the surface of in PDX tumor cell lines (PanCanII-1, 2, 3) by flow cytometry. VIP is expressed by a human pancreatic cell line (Panc1) and PDX tumor cell lines. Experiments indicates that anti-Muc16CD CAR T cells are capable of cytotoxic function against Muc16CD-expressing PDX tumor cells lines (FIG. 3).

### Validation of VIPR Antagonist-Secreting CAR T Cells

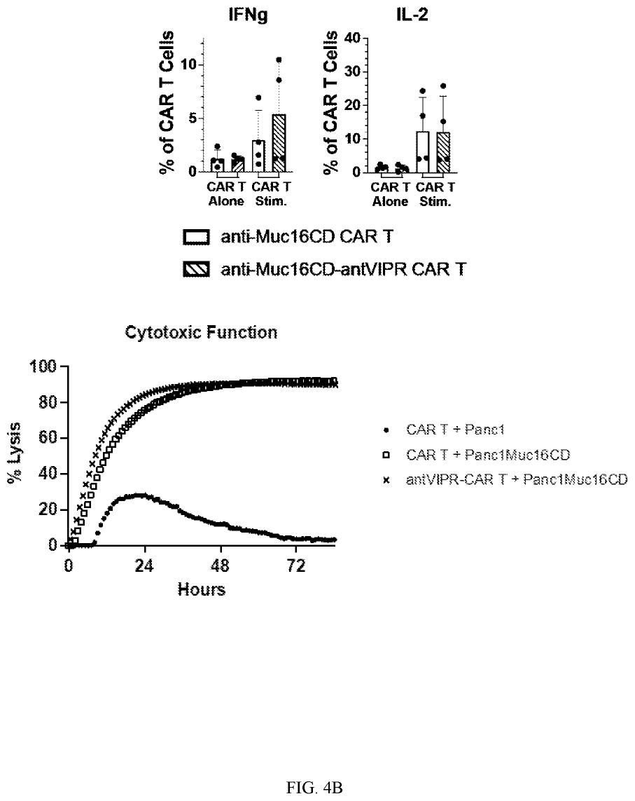

Human CAR T cells can be co-modified to secrete VIPR antagonist peptides. CAR constructs were prepared encoding Muc16-CD targeted VIPR antagonist-secreting CAR. Human primary T cells were transduced with anti-Muc16CD-antVIPR CAR. VIPR antagonist-secreting CART cells are capable of cytotoxic function and cytokine production. VIPR antagonist (˜4 kDa) can be detected in culture supernatant when tagged to Gaussia luciferase (FIGS. 4A and 4B).

### Secreted VIPR Antagonist Alters CAR T Cell Phenotype and Function

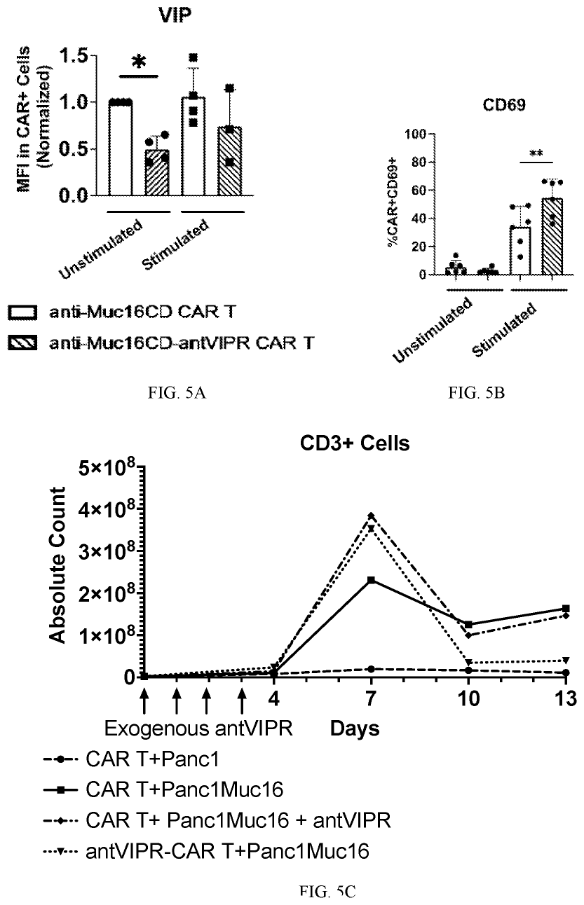

Experiments were performed to determine whether CAR T cell-produced VIPR antagonist peptides are functional. AntVIPR-CAR T cells have reduced VIP and VPAC1 expression at rest. Conversely, antVIPR CAR T cells have increased activation (CD69+) after antigen-stimulation and bulk cell viability after manufacturing. AntVIPR CAR T cells confer a proliferative advantage when expanded in vitro compared to parental CAR T cells similar to parental CAR T cells treated with exogenous antVIPR (FIG. 5A-5C).

### Secreted VIPR Antagonist Improves CAR T Cell Persistence

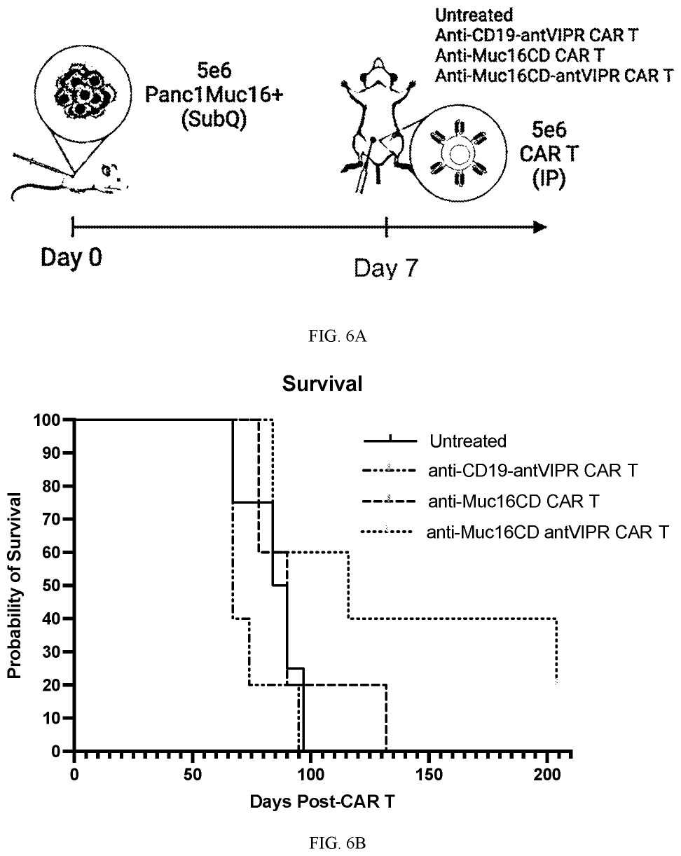

Experiments were performed to determine whether antVIPR-CAR T cells persist in vivo. A schematic of treatment schedule and experimental conditions is shown in FIG. 6A. SCID/Beige mice were engrafted with Panc1-Muc16CD tumors and treated with CAR T cells 5-7 days later intraperitoneally. Human T cells were detectable in anti-Muc16CD-antVIPR CAR T treated mice on days 118 and 204. Kaplan-Meier curve of mice treated with anti-Muc16CD CAR T and anti-Muc16CD-antVIPR CAR T cells. (FIG. 6B)

What is claimed is:

1. A composition comprising
a first nucleic acid sequence encoding a chimeric antigen receptor (CAR) molecule that binds to an antigen and
a second nucleic acid sequence encoding a vasoactive intestinal peptide receptor antagonist.

2. The composition of claim 1, wherein the first nucleic acid sequence and second nucleic acid sequence are disposed on a single nucleic acid molecule.

3. The composition of claim 2, wherein the single nucleic acid molecule encodes a protein that has the following arrangement in an N- to C-terminal orientation: the chimeric antigen receptor, a linker, and the vasoactive intestinal peptide receptor antagonist.

4. The composition of claim 3, wherein the linker is a self-cleaving peptide.

5. The composition of claim 4, wherein the self-cleaving peptide is selected from a P2A site, a T2A site, an E2A site, or an F2A site.

6. The composition of claim 1, wherein the vasoactive intestinal peptide receptor antagonist is a hybrid peptide of neurotensin and a VIP Receptor antagonist consisting of an N-terminal KPRRPY (SEQ ID NO:2).

7. The composition of claim 1, wherein the vasoactive intestinal peptide receptor antagonist is
(SEQ ID NO: 1)
KPRRPYTDNYTRLRKQMAVKKYLNSILN (VIP-hyb).

8. The composition of claim 1, wherein the vasoactive intestinal peptide receptor antagonist has of the amino acid of K P R R P Y X¹ X² N X³ T X⁴ L R K Q X⁵ A V X⁶ K Y X⁷ N X⁸ I L N (SEQ ID NO: 11), wherein X¹ is A or any amino acid; X² is V or any amino acid; X³ is C or any amino acid; X⁴ is S or any amino acid; X⁵ is I or any amino acid; X⁶ is N or any amino acid; X⁷ is M or any amino acid; X⁸ is I or any amino acid.

9. The composition of claim 1 wherein the vasoactive intestinal peptide receptor antagonist has any of the following amino acid sequences
(SEQ ID NO: 3)
KPRRPYADNYTRLRKQMAVKKYLNSILN (Ant-001),
(SEQ ID NO: 4)
KPRRPYTVNYTRLRKQMAVKKYLNSILN (Ant-002),
(SEQ ID NO: 5)
KPRRPYTDNCTRLRKQMAVKKYLNSILN (Ant-003),
(SEQ ID NO: 6)
KPRRPYTDNYTSLRKQMAVKKYLNSILN (Ant-004),
(SEQ ID NO: 7)
KPRRPYTDNYTRLRKQIAVKKYLNSILN (Ant-05),
(SEQ ID NO: 8)
KPRRPYTDNYTRLRKQMAVNKYLNSILN (Ant-06),
(SEQ ID NO: 9)
KPRRPYTDNYTRLRKQMAVKKYMNSILN (Ant-07),
or
(SEQ ID NO: 10)
KPRRPYTDNYTRLRKQMAVKKYLNLILN (Ant-08).

10. The composition of claim 1, wherein the vasoactive intestinal peptide receptor antagonist has of the amino acid of
(SEQ ID NO: 12)
KPRRPYTSDYTRLRKQMAVKKYLNLILN (Ant-308).

11. A recombinant vector comprising a nucleic acid encoding a vasoactive intestinal peptide receptor antagonist having of the amino acid of Y X¹ X² N X³ T X⁴ L R K Q X⁵ A V X⁶ K Y X⁷ N X⁸ I L N (SEQ ID NO: 11), wherein X¹ is A or any amino acid: X² is V or any amino acid; X³ is C or any amino acid; X⁴ is S or any amino acid: X⁵ is I or any amino acid; X⁶ is N or any amino acid: X⁷ is M or any amino acid: X⁸ is I or any amino acid in operable combination with a heterologous promotor.

12. The recombinant vector of claim 11 which is a viral vector, retroviral vector, or lentiviral vector.

13. An immune cell comprising a nucleic acid or recombinant vector comprising a nucleic acid encoding a vasoactive intestinal peptide receptor antagonist having of the amino acid of Y X1 X2 N X3 T X4 L R K Q X5 A V X6 K Y X7 N X8 I L N (SEQ ID NO: 11), wherein X1 is A or any amino acid; X2 is V or any amino acid; X3 is C or any amino acid: X4 is S or any amino acid: X5 is I or any amino acid: X6 is N or any amino acid: X7 is M or any amino acid: X8 is I or any amino acid.

14. A population of immune cells of claim 13 of which more than 15% of the immune cells comprise a nucleic acid or recombinant vector encoding a vasoactive intestinal peptide receptor antagonist having of the amino acid of Y X¹ X² N X³ T X⁴ L R K Q X⁵ A V X⁶ K Y X⁷ N X⁸ I L N (SEQ ID NO: 11), wherein X¹ is A or any amino acid; X² is V or any amino acid; X³ is C or any amino acid: X⁴ is S or any amino acid: X⁵ is I or any amino acid: X⁶ is N or any amino acid: X⁷ is M or any amino acid: X⁸ is I or any amino acid.

15. The population of immune cells as in claim 14 selected from T cells or natural killer cells.

16. A method of treating cancer or other immune cell disease or condition comprising,
contacting, inserting, or transfecting the immune cells with a nucleic acid or recombinant vector encoding a chimeric antigen receptor and encoding vasoactive intestinal peptide receptor antagonist having of the amino acid of Y X¹ X² N X³ T X⁴ L R K Q X⁵ A V X⁶ K Y X⁷ N X⁸ I L N (SEQ ID NO: 11), wherein X¹ is A or any amino acid; X² is V or any amino acid: X³ is C or any amino acid: X⁴ is S or any amino acid: X⁵ is I or any amino acid: X⁶ is N or any amino acid: X⁷ is M or any amino acid: X⁸ is I or any amino acid, as providing immune cells expressing the chimeric antigen receptor and vasoactive intestinal peptide receptor antagonist; and
administering an effective amount of chimeric antigen receptor and vasoactive intestinal peptide receptor antagonist expressing immune cells to a subject in need thereof.

17. The method of claim 16, wherein isolating immune cells is from the subject diagnosed with cancer or other immune cell disease or condition.

18. The method of claim 16, wherein the cancer is a hematological cancer, solid tissue cancer, tumor, or metastatic cancer.

19. The method of claim 16, wherein the cancer is pancreatic ductal adenocarcinoma (PDAC).

20. The method of claim 16, wherein the immune cells are administered in combination with another chemotherapy agent.
